(19) United States
(12) Patent Application Publication
(10) Pub. No.: US 2014/0303737 A1
(43) Pub. Date: Oct. 9, 2014

(54) PHARMACEUTICAL COMPOSITIONS COMPRISING LUBRICANTS FOR PREVENTING OR REDUCING ASEPTIC LOOSENING IN A SUBJECT

(71) Applicant: DANMARKS TEKNISKE UNIVERSITET, Lyngby (DK)

(72) Inventor: Seunghwan Lee, Charlottenlund (DK)

(21) Appl. No.: 14/356,524

(22) PCT Filed: Nov. 5, 2012
(86) PCT No.: PCT/DK2012/050408

§ 371 Date: May 6, 2014

Related U.S. Application Data
(60) Provisional application No. 61/557,049, filed on Nov. 8, 2011.

(30) Foreign Application Priority Data
Nov. 8, 2011 (EP) 11188247.8

(51) Int. Cl. A61L 27/50; A61L 27/18; A61F 2/30

(52) U.S. Cl. CPC A61L 27/50; A61F 2/30; A61L 27/18
USPC 623/18.11; 523/115

(57) ABSTRACT

The present invention relates to pharmaceutical compositions comprising one or more amphiphilic polymer lubricants for use in association with artificial orthopaedic implants. Additionally, the invention relates to medical use of the lubricants of the invention in connection with conditions associated with artificial orthopaedic implants. The invention furthermore relates to artificial joint implants comprising the polymer lubricants according to the invention and methods for preparing such implants.

### TECHNICAL FIELD OF THE INVENTION

The present invention relates to artificial orthopaedic implants. In particular the present invention relates to compositions and methods for improving the tribological properties of artificial orthopaedic implants post-surgically.

### BACKGROUND OF THE INVENTION

Orthopaedic implants are designed to operate under mechanical stress, in particular, tribological stress at the interface of the acetabular cup and femoral head. Poor tribological properties, for instance, wear particles of Ultra-high Molecular Weight Polyethylene (UHMWPE) acetabular cup, are known to be a cause for the failure of the orthopaedic implants, giving rise to medical conditions in the subject such as aseptic loosening and dislocation of the implants. Thus, it is critical for the materials employed for orthopaedic implants to display excellent tribological properties. However, it is well known that the lubricating performance of the currently prevailing implant materials is far less efficient than natural synovial joints, and their life-time is currently limited to about 10-15 years in best case. Hereafter, the implants will have to be replaced by surgical procedure, which is not only costly and very unpleasant for the patient, but also confer risk of serious post-surgical side effects. For younger patients this means that they will have to undergo implant replacement surgery several times during their active lives.

Efforts to improve tribological properties of orthopaedic implants have been exercised nearly exclusively through the development and/or application of new implant materials, as well as modifying the surfaces of the implant material prior to surgery. As an example, U.S. Pat. No. 7,785,372 relates to an artificial joint member made of a polymeric material for use in surgical joint replacements, where the artificial joint member is coated with lubricants. Although the coating of artificial implants with lubricants may have a certain lubricating effect for a limited time, stress and wear at the interface of the acetabular cup and femoral head of the implant will relatively quickly reduce the tribological properties of the artificial implant as the surface coating diminish.

Honda et al discloses 3% polyethylene glycol (PEG) dissolved in water as artificial joint lubricity agent for reducing the wear of UHMWPE (Honda et al. Development of artificial intra-articular polyethylene glycol (PEG) lubricant for survival of total knee joint patient, 2011 11th IEEE International Conference on Bioinformatics and Bioengineering).

Thus, there is need for means for improving the tribological properties of artificial implants so that the implants will only wear to a very limited extent, resulting in implants that are able to maintain functionality for many years.

### SUMMARY OF THE INVENTION

The present invention provides pharmaceutical compositions comprising lubricants, which may post-surgically be injected into artificial implants, or to the surroundings of the artificial implants, resulting in vastly improved tribological conditions at the interface of the acetabular cup and femoral head of the implants. The pharmaceutical compositions may be injected several times over many years, ultimately resulting in artificial implants that have permanently improved tribological properties and therefore may last for many years, which for the majority of patients may be their entire lives. Since the compositions and lubricants of the present invention may be injected any time after surgery, not only patients who will receive the joint replacements in the future, but also those who already have received joint replacements will benefit from the invention.

An object of the present invention is to provide injectable compositions with lubricating effect towards artificial orthopaedic implants, so that the tribological properties of said implants are improved.

Thus, an aspect of the present invention relates to a pharmaceutical composition comprising one or more amphiphilic polymer lubricants and at least one pharmaceutically acceptable carrier, for the prevention or reduction of aseptic loosening in a subject comprising an artificial joint implant.

Another aspect of the present invention relates to an artificial joint implant comprising
a first artificial element 1 and a second artificial element 2 constituting an artificial joint arranged for being implanted in a subject;
a lubricant compartment 3 between said first element and said second element, said lubricant compartment comprising one or more amphiphilic polymer lubricants in a liquid state serving to reduce tribological friction and wear of said implant.

A further aspect of the present invention relates to a method for preparaing an artificial joint implant according to the present invention comprising
providing an artificial joint implant comprising
   a first artificial element 1 and a second artificial element 2 constituting an artificial joint arranged for being implanted in a subject;
   a lubricant compartment 3 between said first element and said second element;
positioning one or more amphiphilic polymer lubricants in a liquid state in the lubricant compartment (3); and
providing an artificial joint implant according to the present invention.

### BRIEF DESCRIPTION OF THE FIGURES

FIG. 1

FIG. 1 shows the effect of lubricant effect of P105 with the final concentration 1% according to the Pin-On-Disk Tribometry method.

FIG. 2

Coefficient of friction (COF) vs. number of rotations for the sliding contacts between CoCrMo pin and UHMWPE disk in serum by lubrication with F127 as determined by Pin-on-Disk Tribometry. The arrow indicates the injection of 1 ml of F127 20% into 2 ml serum solution. The final concentration of F127 in serum is 6.7%.

FIG. 3

The % reduction of the copolymers, F127, F108, P105, and F68, in variation of concentration by injection of 1 ml of the lubricants into 2 ml calf serum where the sliding contacts between CoCrMo/UHMWPE is taking place. The numbers after the names of the copolymers represent the concernation prior to injection. The final concentrations in serum are diluted to ⅓ of the original values.

FIG. 4

The % reduction in COF of the homopolymers, PEO 5k, PEO 600k, and PAA 5.1 k, in variation of concentration by injection of 1 ml of the lubricants into 2 ml serum where the sliding contacts between CoCrMo/UHMWPE is taking place. The numbers after the names of the copolymers represent the concernation prior to injection. The final concentrations in serum are diluted to ⅓ of the original values.

FIG. 5

COF vs. number of rotations for the sliding contacts between CoCrMo pin and UHMWPE disk in calf serum by lubrication with biological polymers, Bovine Submaxillary Mucin (BSM), Hayluronic Acid (HA), Bovine Serum Albumin (BSA), and Alginic Acid (AA) as determined by Pin-on-Disk Tribometry. The concentrations of BSM, HA, BSA, and AA were 5%, 1%, 10%, and 5%, respectively, prior to injection. The arrow indicates the injection of 1 ml of the lubricant into 2 ml serum in which the sliding contacts between CoCrMo and UHMWPE is taking palce.

FIG. 6

COF vs. number of rotations for the sliding contacts between CoCrMo pin and UHMWPE disk in serum by lubrication with F127 as determined by Pin-on-Disk Tribometry. It is noted that except for the temperature, 37° C., the experimental conditions are identical with those of FIG. 2.

FIG. 7

COF vs. number of rotations for the sliding contacts between CoCrMo pin and UHMWPE disk in HA-serum by lubrication with F127 20% (A) and F127 10% (B) as determined by Pin-on-Disk Tribometry. It is noted that except for the presence of HA at 3.5 mg/ml, the experimental conditions are identical with those of FIG. 2 and FIG. 3.

FIG. 8

COF vs. number of rotations for the sliding contacts between CoCrMo pin and UHMWPE disk in serum by lubrication with or without F127 10% as determined by Pin-on-Disk Tribometry for 100,000 rotations (=ca. 3,000 m in total sliding length).

FIG. 9

COF vs. number of rotations for the sliding contacts between “aged CoCrMo pin and UHMWPE disk in serum for 4 weeks” by lubrication with or without F127 10% as determined by Pin-on-Disk Tribometry for about 1 hour.

FIG. 10

MTT assay results on the copolymers, F127, F108, P105, and F68. Intesities of purple formazan upon reduction by mitochondria of living cells (fibroblast) in the presence of the external lubricants. The first column is for PBS control (=no external lubricants), and the data for the external lubricant are normalized to the control. The concentrations of the copolymers were 5% (corresponding to 15% of the lubricants prior to injection) and the test duration was 24 hours.

FIG. 11

FIG. 11 shows illustrative examples of artificial joint implants according to the present invention. 1 and 2 show the solid parts of the implant and 3 illustrates a position of the lubricant compartment(s). (a) illustrates a hip phrosthesis and (b) illustrates a knee phrostesis.

FIG. 12

FIG. 12 shows how lubricants according to the present invention may be positioned in an artificial joint implant before or after positioning of the implant in a subject. 1 and 2 show the solid parts of the implant, 3 illustrates a position of the lubricant compartment(s) and 4 illustrates injection of the lubricants by a syringe.

FIG. 13

FIG. 13 illustrates the formula of triblock copolymers according to the invention.

### DETAILED DESCRIPTION OF THE INVENTION

The present inventor has discovered that certain types of lubricants may function as lubricants for the prevention of asceptic looseing in subjects having an artificial joint implant. Furthermore, such lubricants may be added to the artificial joint implant both before and after implantation. Honda el al. discloses that PEG (or PEO) dissolved in water may function as a lubricant in artificial joints. However Honda et al. is silent in respect of supplying the lubricant to the artificial joint after the implant has been positioned in a subject (e.g. human). Furthermore, In Honda et al. PEG is dissolved in water which does not resemble synovial fluid which will normally be present after implantation. In example 4 the lubrication effect of different lubrication is tested in model synovial fluid.

### Pharmaceutical Composition

The present invention relates in one aspect to a pharmaceutical composition comprising one or more lubricants and at least one pharmaceutically acceptable carrier.

The term “lubricant” pertains to any substance that is able to reduce friction between two or more moving surfaces. In particular, the term “lubricant” does in the present context relate to any substance that is able to reduce friction in an artificial orthopaedic implant, thereby improving the tribological properties of said implant. It follows that the lubricant is biocompatible in the sense that the lubricant elicits little or no immune or toxic response in the subject to which it is administrated.

An aspect of the present invention relates to a pharmaceutical composition comprising one or more amphiphilic polymer lubricants and at least one pharmaceutically acceptable carrier, for the prevention or reduction of aseptic loosening in a subject comprising an artificial joint implant. Aseptic loosening, the most common medical conditions of long-term failure in artificial joint replacements (especially hip and knee replacements). Aseptic loosening occurs when normal wear on a prosthetic joint produces microscopic debris particles that trigger an immune system response. This immunologic activity mediates osteolysis, which loosens the bond between the implant and bone. Thus, the pharmaceutical composition may be considered to provide prophylaxtic treatment.

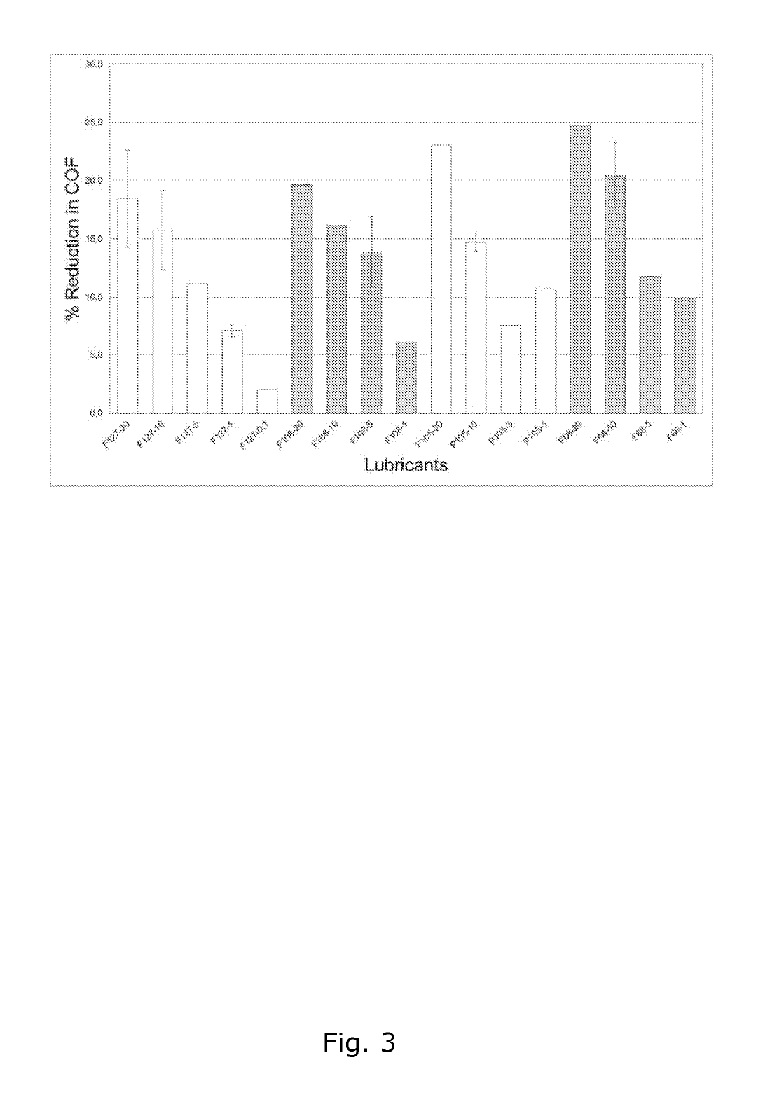

The inventor has discovered that amphiphilic polymer lubricants functions superior to biological polymers (see FIGS. 3-5) and are well tolerated by living cells (see example 8). The pharmaceutical composition functions by modifying the synovial fluid present “in” the implanted artificial joint implant.

In the present context “amphiphilic” is to be understood as having a potential to adsorb both hydrophilic and hydrophobic surfaces.

Thus, a main object of the present invention is therefore to provide compositions comprising lubricants that may be delivered for example by injection into, or to the proximity of an artificial orthopaedic implant (artificial joint implant) post-surgically such as into or in the proximity of the interface of the acetabular cup and femoral head. However, in certain embodiments of the present invention, the compositions comprising lubricants may also be utilised in coating the artificial orthopaedic implant prior to surgery. Alternatively the lubricant is positioned in a liquid state in the artificial orthopaedic implant (artificial joint implant) prior to surgery.

The effect on aseptic loosening may also be described in other ways. Thus another aspect of the invention relates to a pharmaceutical composition comprising one or more amphiphilic polymer lubricants and at least one pharmaceutically acceptable carrier, for reducing or prevention the risk of toxication or inflammation in a subject comprising an artificial joint implant, by preventing or reducing of the release of debris from the artificial joint implant.

Yet an aspect of the invention relates to a pharmaceutical composition comprising one or more amphiphilic polymer lubricants and at least one pharmaceutically acceptable carrier, for the prevention of artificial joint implant replacement surgery or for prolonging the period before an artificial joint implant replacement surgery is needed in a subject comprising an artificial joint implant. As mentioned above the life-time of artificial joint implant is currently limited to about 10-15 years in best case. Hereafter, the implants will have to be replaced by surgical procedure, which is not only costly and very unpleasant for the patient, but also confer risk of serious post-surgical side effects. By extending the life-time of the implant a patient may completely avoid having to have the implant replaced. This problem is increasing due to the increased life-time of people and an increasing number of younger people getting artificial joint implants.

The term “pharmaceutically acceptable” refers in the present context to molecular entities and compositions that do not produce an allergic, toxic, or otherwise adverse reaction when administered to an animal, particularly a mammal, and more particularly a human. Pharmaceutical acceptable carriers include any and all solvents, dispersion media, coatings, antibacterial and antifungal agents, stabilizers, isotonic and absorption delaying agents and the like. The use of such media and agents for pharmaceutical active substances is well known in the art.

Exemplary liquid carriers include water, oils, buffers etc., and mixtures hereof. Liquid carriers are used in preparing solutions, suspensions, emulsions, elixirs and pressurized compositions. The active ingredient, in this case the one more or lubricant, can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, suspending agents, thickening agents, colours, viscosity regulators, stabilizers or osmo-regulators. Suitable examples of liquid carriers for parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate.

The lubricants according to present invention may be lubricants of any origin. Hence, in an embodiment the present invention relates to a pharmaceutical composition comprising one or more lubricants which are selected from one or more of the groups of lubricants consisting of synthetic lubricants and natural lubricants, preferably synthetic lubricants.

In the present context a “natural lubricant” or “biological lubricant” refers to any naturally occurring lubricant and its derivatives that may be isolated or extracted from any natural occurring source, such as animals, plants, moss, microorganisms including yeast and bacteria, as well as viruses. On the other hand, synthetic lubricants relates to any lubricant and its derivatives that is not naturally occurring, but may be man-made e.g. by chemical synthesis or by genetic engineering and in vitro expression.

As described above, an object of the present invention is to provide lubricants for artificial orthopaedic implants that are effective post-surgically when the artificial joint has been implanted. Hence, the lubricants of the present invention are effective when present in an environment that resembles that of the normal synovial joint. Thus, in another embodiment, the present invention pertains to a pharmaceutical comprising one or more lubricants, where said one or more lubricants are capable of reducing (delaying) the tribological wear of an orthopaedic implant in an environment comprising a synovial fluid.

It is within the scope of the present invention that the one or more lubricants may be any chemical entity. However, the lubricants of the invention are preferably polymeric molecules. Hence, the present invention relates in an embodiment to a pharmaceutical composition comprising one or more lubricants, where said one or more lubricants are amphiphillic copolymers. An advantage of amphiphilic copolymers may be especially pronounced when one of the implant surfaces is a UHMWPE surface, which is hydrophobic and interfacing water/fluid is hydrophilic. Thus amphiphilic copolymers can readily reside at the interface.

### Administration

As described earlier, the pharmaceutical compositions and lubricants according to the present invention are useful for administration by injection however other means of administration may be used. The frequency of administration as well as the amount of pharmaceutical composition or lubricant to administer per administration may depend on both the physical-chemical nature of the composition or lubricant to be administered, the size and type of artificial orthopaedic implant (both with respect to where the implant is to be inserted and to what material combinations the implant comprises of), as well as on the daily working load the implant is subjected to.

For the lubricants to work efficiently the lubricant should of course be positioned optimally in the implant. Thus, in an embodiment the pharmaceutical composition is administered to a lubricant compartment 3 between a first artificial element 1 and a second artificial element 2 of an artificial joint implant before or after the joint implant is positioned in the subject. As also shown in the figures the lubricant should be positioned at the interface between the first and the second artificial element. The lubricants of the present invention may thus be applied to the artificial orthopaedic implant or to the proximity of the artificial orthopaedic implant by any suitable method, preferably by injection. Hence, the present invention also relates to a pharmaceutical composition comprising one or more lubricants, where said composition is formulated for administration by injection.

In normal joint implant natural synovial fluid functions as lubricants in at the interface between the moving elements of the implant. Thus, the lubricants according to the present invention need to function in the presence of synovial fluid. Thus, in an embodiment the lubricant compartment is in fluidic contact with synovial fluid.

The pharmaceutical composition may be administered to the subject in different ways. In an embodiment the pharmaceutical composition is formulated for administration by injection. Other types of administration may also be envisioned by the skilled person, e.g. from an internal chamber in the implant which may be reloaded from an external source, by oral administration or by dermal administration.

### Concentration of the Lubricants

The concentration of the lubricants in the pharmaceutical composition may also be optimized. Thus, in an embodiment the amphiphillic lubricants are present in a total concentration 1-20% (weight/volume), such as 1-15%, such as 1-10%, such as 1-5%, such as 5-20%, such as 10-20%. The results are presented in example 4, showing that overall a higher concentration is more efficient.

### Type of Amphiphilic Polymer Lubricants

The amphiphilic polymer lubricants according to the present invention may be further specified in regard of their structure. Thus in an embodiment the one or more lubricants comprises poly(ethylene oxide) (PEO), poly(propylene oxide) and/or polyacrylic acid (PAA) polymers. As also shown in example 4 these types of polymers all functions better than the natural polymers. Table 1 shows details of some of the lubricants according to the present invention.

TABLE 1
Details of lubricants according to the present invention:

| Name | Synonyms | Specific details |
|---|---|---|
| Copolymers | | |
| F127 | poly(ethylene oxide)-block-poly | Average |
| | (propylene oxide)-block-poly | molecular |
| | (ethylene oxide) | weight: 12600 |
| | (PEO-b-PPO-b-PEO) | Da |
| | | PEO₁₀₀-b-PPO₆₅- |
| | | b-PEO₁₀₀ |
| F108 | Poly(ethylene glycol)-block- | Average |
| | poly(propylene glycol)-block- | molecular |
| | poly(ethylene glycol) | weight: 11680 |
| | PEG-PPG-PEG | Da |
| | | PEO₁₃₃-b-PPO₅₀- |
| | | b-PEO₁₃₃ |
| 105 | Poly(ethylene glycol)-block-poly | Average |
| | (propylene glycol)-block-poly | molecular |
| | (ethylene glycol), Poly(propylene | weight: 6500 |
| | glycol)-block-poly(ethylene glycol)- | Da |
| | block-poly(propylene glycol) | PEO₃₇-b-PPO₅₆- |
| | (PEO-b-PPO-b-PEO) | b-PEO₃₇ |
| F68 | Polyoxyethylene-polyoxypropylene | Average |
| | block copolymer, Poly(ethylene | molecular |
| | glycol)-block-poly(propylene | weight: 8400 |
| | glycol)-block-poly(ethylene glycol) | Da |
| | (PEO-b-PPO-b-PEO) | PEO₆₇-b-PPO₂₀- |
| | | b-PEO₆₇ |
| Homopolymers | | |
| PEO 5k | Poly(ethylene glycol) | Average |
| | | molecular |
| | | weight: 5000 |
| | | Da |
| PEO 600k | Poly(ethylene glycol) | Average |
| | | molecular |
| | | weight: 600000 |
| | | Da |
| PAA 5.1k | Poly(polyacrylic acid) | Average |
| | | molecular |
| | | weight: 5100 |
| | | Da |

It is noted that “Pluronic” are also known as “Poloxamer” or “Kolliphor” as well.

In the present context the terms “amphiphillic copolymers” or “amphiphillic (co)polymers” refer to heteropolymeric molecules that comprise both hydrophilic and hydrophobic monomeric species, also known as residues or units. There are different types of amphiphillic copolymers, including “alternating copolymers” where the different monomeric units (e.g. A and B) are linked in a systematic alternating pattern, “block copolymers”, also known as “amphiphillic block copolymers” or simply “AB” where the different monomeric units (e.g. A and B) are located in separated homopolymeric blocks that are linked together, “random copolymers” where the different monomeric units (e.g. A and B) are linked in a randomized pattern and “graft copolymers” which is a branched copolymer in which the side chains (e.g. comprising B monomeric units) are structurally distinct from the main chain (e.g. comprising A monomeric units). Below are presented structural examples of the different amphiphillic copolymers:

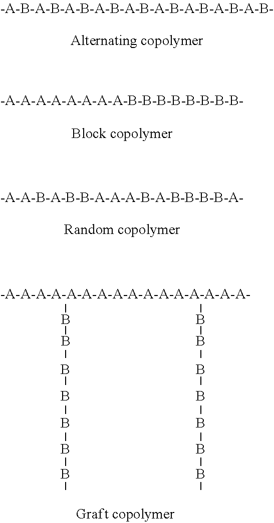

Block copolymers are typically named according to the number of homopolymeric blocks they contain. Hence the above shown example

“A-A-A-A-A-A-A-A-B-B-B-B-B-B-B-B”

is a “Di-block copolymer” comprising an “A” homopolymeric block and a “B” homopolymeric block linked together.

“Tri-block copolymers” are copolymers comprising three homopolymeric blocks that are linked together. All three blocks may comprise different monomeric units, but two of the blocks, both ends, may comprise the same monomeric unit. Hence, the structure

“-A-A-A-A-A-A-A-A-B-B-B-B-B-B-B-B-C-C-C-C-C-C-”

is known as an “A-B-C” tri-block copolymeric form comprising three different monomeric units (i.e. A, B and C), while the structure

“-A-A-A-A-A-A-A-A-B-B-B-B-B-B-B-B-A-A-A-A-A-A-A-”

is an “A-B-A” tri-block copolymeric form comprising only two different monomeric units (i.e A and B). In particular, an “A-B-A” or “B-A-B” polymeric form may in the present context be regarded as the tri-block polymeric form of the di-block copolymer “A-B” or “B-A”.

Examples of tri-block copolymers are PEO-PPO-PEO, and more specifically F127, F108, F68, and P105, according to the molecular weight and the ratio between PEO and PPO blocks (see table 1). In a preferred embodiment the lubricant is a tri-block copolymers such as F127, F108, F68, and P105 and the join implant comprises UHMWPE.

The corresponding nomenclature are used to name block copolymers comprising more homopolymeric blocks (e.g. tetra-block copolymers, penta-block copolymers, hexa-block copolymers, etc.).

It is within the scope of the present invention that the lubricants of the present invention may be any type of block copolymer, and that the homopolymeric blocks may be arranged in any possible order.

Thus, in an embodiment the present invention relates to a pharmaceutical composition comprising one or more lubricants, where the one or more lubricants are independently selected from the group consisting of poly(ethylene oxide)-based block copolymers, Carbohydrate-based block copolymers, and Poly(vinylpyrrolidone)-based block copolymers.

In another embodiment the present invention relates to a pharmaceutical composition comprising one or more lubricants, where said one or more lubricants are independently selected from the group of di-block copolymers consisting of poly(propylene oxide)-b-poly(ethylene oxide), poly(butadiene(1,2 addition))-b-poly(ethylene oxide), poly(butadiene(1,4 addition))-b-poly(ethylene oxide), poly(ethylene oxide)-b-poly(s-caprolactone), poly(ethylene oxide)-b-poly-Lactide, poly(ethylene oxide)-b-poly(methyl acrylate), -poly(ethylene oxide)-b-Poly(Isobutylene), poly(ethylene oxide)-b-poly-Lactide, poly(ethylene oxide)-b-poly(acrylic acid), poly(ethylene oxide)-b-poly(acrylamide), poly(ethylene oxide)-b-poly(methyl acrylate), poly(ethylene oxide)-b-poly(N-isopropylacrylamide), poly(ethylene oxide)-b-poly(dimethylsiloxane), poly(butadiene(1,2 addition))-b-poly(acrylic acid), poly(butadiene(1,4 addition))-b-poly(acrylic acid), poly(Isobutylene)-b-poly(acrylic acid), Poly(vinyl pyrrolidone)-b-Poly(D/L-Lactide), and any tri-block copolymeric forms (A-B-A or B-A-B) of the above mentioned di-block copolymers, and in any possible combination.

In yet another embodiment the present invention relates to a pharmaceutical composition comprising one or more lubricants, where said one or more lubricants are independently selected from the group of di-block copolymers consisting of poly(propylene oxide)-b-poly(ethylene oxide), poly(butadiene(1,2 addition)-b-poly(ethylene oxide), poly(butadiene(1,4 addition))-b-poly(ethylene oxide), and any tri-block copolymeric forms (A-B-A or B-A-B) of the above mentioned di-block copolymers, and in any possible combination.

In a further embodiment the present invention relates to a pharmaceutical composition comprising one or more lubricants, where said one or more lubricants are independently selected from the group of di-block copolymers consisting of poly(ethylene oxide)-b-poly(s-caprolactone), poly(ethylene oxide)-b-poly-Lactide, poly(ethylene oxide)-b-poly(methyl acrylate), poly(ethylene oxide)-b-Poly(Isobutylene), poly(ethylene oxide)-b-polyLactide, poly(ethylene oxide)-b-poly(acrylic acid), poly(ethylene oxide)-b-poly(acrylamide), poly(ethylene oxide)-b-poly(methyl acrylate), poly(ethylene oxide)-b-poly(N-isopropylacrylamide), and any tri-block copolymeric forms (A-B-A or B-A-B) of the above mentioned di-block copolymers, and in any possible combination.

In another embodiment the present invention relates to a pharmaceutical composition comprising one or more lubricants, where said one or more lubricants are independently selected from the group of di-block copolymers consisting of poly(ethylene oxide)-b-poly(dimethylsiloxane), poly(butadiene(1,2 addition))-b-poly(acrylic acid), poly(butadiene(1,4 addition))-b-poly(acrylic acid), poly(Isobutylene)-b-poly(acrylic acid), Poly(vinyl pyrrolidone)-b-Poly(D/L-Lactide), and any tri-block copolymeric forms (A-B-A or B-A-B) of the above mentioned di-block copolymers, and in any possible combination.

It is within the scope of the present invention that the different lubricants described herein above may be selected independently from any of the different groups described above, and that these different lubricants from different groups may be combined in any possible way and in any possible order.

The different types of polymeric lubricants according to the present invention all have different molecular weight (see also table 1). Thus, in another embodiment the one or more lubricants has a molecular weight in the range 3000 Da to 1,000,000 Da, such as in the range 3000 Da to 500,000 Da, such as in the range 3000 Da to 100,000 Da, such as in the range 3000 Da to 50,000 Da, such as in the range 3000 Da to 30,000 Da, such as in the range 3000 Da to 15,000 Da, such as in the range 5000 Da to 30,000 Da, such as in the range 10,000 Da to 30,000 Da, or such as in the range 10,000 Da to 15,000 Da.

The lubricants according to the present invention may be further specified. Thus, in an embodiment the one or more amphiphillic lubricants are amphiphillic co-polymers. In yet an embodiment the one or more amphiphillic lubricants are poly(ethylene oxide)-based block copolymers. In yet another embodiment the amphiphillic lubricant is a poly(ethylene oxide)-block-poly(propylene oxide)-block-poly(ethylene oxide) (PEO-PPO-PEO) triblock copolymer. In the example section positive lubrication have been tested for F127, F108, F68, and P105. Thus, in an embodiment the amphiphillic lubricants is selected from the group consisting of F127, F108, F68, and P105. Preferably the lubricant is F127 or F108.

The lubricants according to the invention may also be homopolymers. Thus, in an embodiment the amphiphillic lubricant is a homopolymer. In yet an embodiment the homopolymer is selected from the group consisting of ethylene oxide polymers (PEO) and polyacrylic acid (PAA) polymers. PEO is often described as to have “amphiphilicity” i.e. PEO sticks to both hydrophilic and hydrophobic surfaces. Thus, PEO can be called an “amphiphilic” polymer. PEO is not “co”polymer since it is made of ethylene oxide (EO) only, and belongs to the category of homopolymer. In another embodiment the homopolymer is selected from the group consisting of PEO 5k, PEO 600k, and PAA 5.1 k (for further details see table 1). In example 4 different homopolymers have been tested.

Since both triblock and homopolymers have shown to be effective lubricants, it is reasonable to assume that the same will be the case for di-block copolymers. Thus, in an embodiment the amphiphillic lubricant is a di-block copolymer.

The lubricants according to the invention may be further defined by their functionality. As already described, the compositions and lubricants of the present invention are capable of improving the tribological properties of the artificial orthopaedic joint to which they are applied. In particular, the lubricants of the invention are able to reduce friction between the acetabular cup and femoral head of the artificial joint as simulated by the Pin-On-Disk Tribometry method described in the Examples herein below, which measures the coefficient of friction. The “coefficient of friction” (COF), also known as a “frictional coefficient”’ or “friction coefficient”, is a dimensionless scalar value which describes the ratio of the force of friction between two bodies and the force pressing them together. The coefficient of friction is determined by measuring the torque force by strain gauge during loaded sliding contacts between the pin and the disk. Load and speed can be varied, and the coefficient of friction is obtained from friction force/Load. Thus, a reduction in coefficient of friction according to the Pin-On-Disk Tribometry method, is indicative for that the applied lubricant or lubricants are capable of improving the tribological properties of an artificial orthopaedic joint.

Hence, the present invention does in one embodiment relate to a pharmaceutical composition comprising one or more lubricants, where said composition is able to reduce the coefficient of friction according to the Pin-On-Disk Tribometry method by at least 0.05 in a buffer solution, or at least 0.015 in a calf serum solution.

In another embodiment, the pharmaceutical composition comprising one or more lubricants is able to reduce the coefficient of friction according to the Pin-On-Disk Tribometry method by at least 0.06, such as at least 0.08, e.g. at least 0.10, such as at least 0.12, e.g. at least 0.14, for example at least 0.16, such as at least 0.18, e.g. at least 0.20 in a buffer solution, or by at least 0.015, such as at least 0.025, e.g. at least 0.035, such as at least 0.045. e.g. at least 0.055, for example at least 0.065, such as at least 0.075 in a calf serum solution.

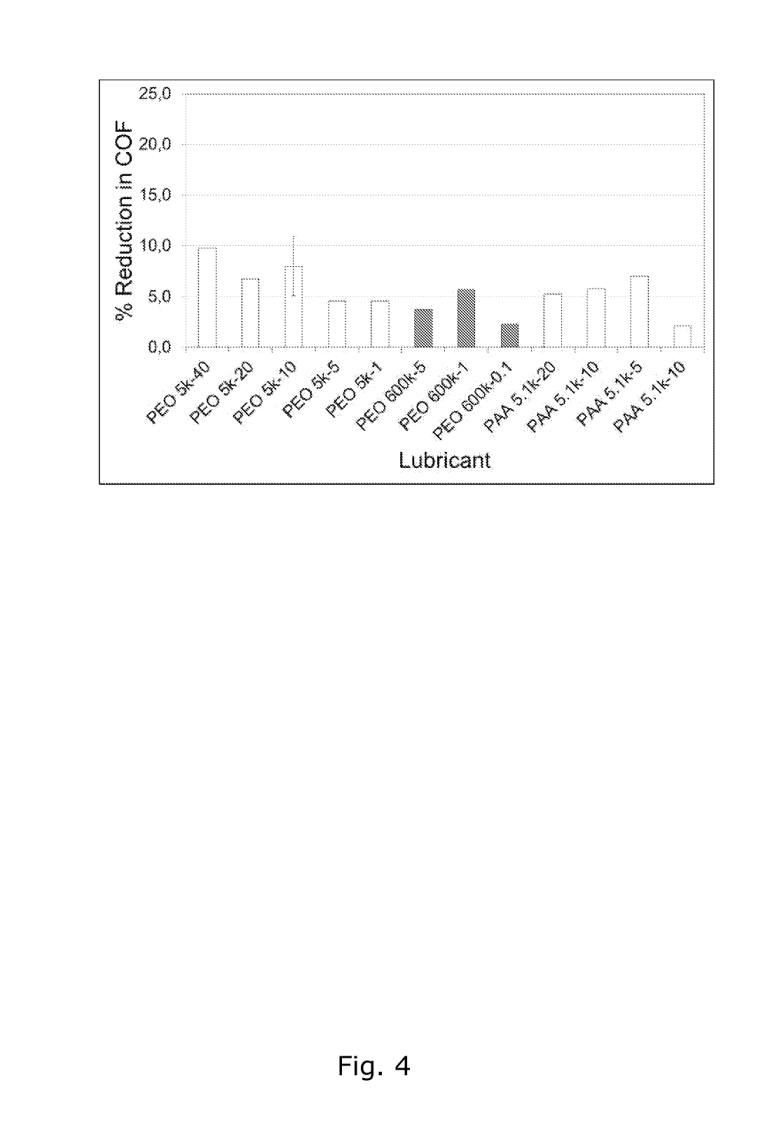

Preferably the reduction is described in percentage. Thus, in yet an embodiment the invention relates to a pharmaceutical composition comprising one or more lubricants, where said composition is able to reduce the coefficient of friction according to the Pin-On-Disk Tribometry method by at least 1%, such as at least 5% such as at least 10%, such as at least 15%, such as at least 20%, such as at least 25%, such as at least 30%. Example 4 and FIG. 4 shows the reduction in COF for different lubricants.

The pharmaceutical composition comprising one or more lubricants according to the invention, is also capable of reducing the wear of the artificial implant as simulated by the Pin-On-Disk Tribometry method as described in the Examples below. Likewise, the pharmaceutical composition comprising one or more lubricants of the present invention are able to reduce friction in and wear of artificial orthopaedic implants. This may be simulated by the Joint Simulator method.

It is within the scope of the invention that the lubricants and compositions of the present invention are capable of reducing friction in and wear of artificial orthopaedic implants according to both the Pin-On-Disk Tribometry method and in calf serum solutions, calf serum solution added with hyaluronic acid. These model systems mimics natural synovial fluid. Thus, in another embodiment the pharmaceutical composition is able to reduce the coefficient of friction according to the Pin-On-Disk Tribometry method by at least 1%, such as at least 5% such as at least 10%, such as at least 15%, such as at least 20%, such as at least 25%, or such as at least 30%.

### Type of Artificial Joint Implant

Suitable artificial orthopaedic implants that may be target of the present invention includes a hip prosthesis, a knee prosthesis, a shoulder prosthesis, a foot and ankle prosthesis, a toe prosthesis, an elbow prosthesis, a hand and wrist prosthesis, a finger prosthesis combinations thereof. Thus, in an embodiment the artificial joint implant is selected from the group consisting of a hip prosthesis, a knee prosthesis, a shoulder prosthesis, a foot and ankle prosthesis, a toe prosthesis, an elbow prosthesis, a hand and wrist prosthesis, and a finger prosthesis.

Artificial orthopaedic implants may be produced in various materials, and different parts of the implant may be produced in different materials. Accordingly, the acetabular cup and femoral head of the implants may be produced in the same material, or alternatively in different materials. Suitable materials of artificial orthopaedic implants that may be target of the present invention includes UHMWPE (Ultra-high Molecular Weight Polyethylene), CoCrMo alloy, alumina ceramic and alumina alloy, in particular Zirconia-Toughened Alumina (ZTA). Thus, in another embodiment the artificial joint implant comprises materials selected from the group consisting of UHMWPE (Ultra-high Molecular Weight Polyethylene), CoCrMo alloy, alumina ceramic and alumina alloy, Zirconia-Toughened Alumina (ZTA) and combinations thereof. It is within the scope of the invention that the above listed materials may be combined in any possible way in the artificial orthopaedic implant, such that the acetabular cup is produced in one material and the femoral head is produced in the same or another material. If the acetabular cup is produced e.g. in UHMWPE and the femoral head is produced e.g. in CoCrMo alloy, such material combination is referred to as a “UHMWPE versus CoCrMo alloy” implant. Suitable material combinations that may be targeted with the compositions and lubricants of the present invention are UHMWPE versus CoCrMo alloy, UHMWPE versus alumina ceramic, CoCrMo alloy versus CoCrMo alloy, alumina ceramic versus alumina ceramic, and CoCrMo alloy versus alumina ceramic.

### Artificial Joint Implant

Artificial orthopaedic implants (artificial joint implant) or artificial orthopaedic joints are mechanical replacements for injured or damaged normal joints. Joints are locations at which two or more bones make contact, and are constructed to allow movement and provide mechanical support. The injured or damaged joint is surgically removed and replaced by implanting the artificial orthopaedic joint which then will, to some extent, restore movement and support at the site of implant. In the present context, the word “orthopaedic implant” is interchangeable with the word “joint”. In yet an embodiment the artificial joint implant is a total joint replacement implant.

Three main joint structures exist: Fibrous joints where the bones are joined by connective tissue, cartilaginous joints where the bones are joined by cartilage, and synovial joints where the bones are not joined directly, but have a synovial cavity and are united by the connective tissue that forms the articular capsule. Fibrous and cartilaginous joints provide little or no movements, while synovial joints provide movement to various degrees. Examples of synovial joints are found several places in the mammal body, e.g. in the hip knee, shoulder, elbow, foot, ankle, toe, hand, wrist and finger.

The synovial joints contain a synovial cavity where a fluid, the synovial fluid, provides the necessary reduction of friction between the articular cartilage of the joint during movement. The synovial fluid is a viscous fluid with yolk-like consistency which contains a number of different molecular components, including hyaluronic acid (hyaluronan) which is a polymer of disaccharides composed of D-glucuronic acid and D-N-acetylglucosamine, and the glycoprotein lubricin (proteoglycan 4).

Under conditions of reduced movement capabilities of the joint, e.g. when a patient is suffering from arthritis, the reduced movement may be caused by increased friction between the articular cartilage of the joint. Therefore it has for many years been known to inject hyaluronic acid or its derivatives into the joint, in an attempt to reduce friction in the articular cartilage, thereby improving the movement capabilities of the joint.

Reduced movement capabilities in artificial orthopaedic joints used as replacements for synovial joints are often associated with stress and wear of the artificial joint, resulting in reduced tribological properties of the artificial joint. In the present context the wording “tribological properties” pertains to the movement/motion capabilities at the interface of two or more opposing solid materials. Hence, reduced tribological properties mean that the artificial joint has reduced movement/motion capabilities, e.g. due to increased friction as a result of wear and/or lack of lubrication. Thus far, dealing with artificial orthopaedic joints with reduced tribological properties to an extent where the artificial joint becomes non-functional due to stress and wear, has been performed by replacing the artificial implant with a new artificial implant. However, this procedure has a number of disadvantages as described above.

The present invention solves this problem by providing compositions and methods for injecting or delivering lubricants into the artificial joint, or to the proximity of the artificial joint, thereby improving the tribological properties and thereby decreasing/delaying wear of the joint, ultimately resulting in greatly prolonged life-time of the artificial joint. Hence the present invention provides an advantageous approach for dealing with artificial joints that have reduced tribological properties, by offering relatively risk-free repetitive post-surgical injections with lubricants or delivery of lubricants, as an alternative to repetitive surgical implant replacements. Further, the present invention has the advantage, that it can be applied to new artificial joints prior to implantation, as well as to artificial joints that have already been implanted in the individual (in situ), which is not the case with existing technology comprising implants with surface coatings.

The role of synovial fluid, and in particular of hyaluronic acid in normal synovial joints, is believed to be associated with regulating the hydrostatic pressure in articular cartilage. Artificial orthopaedic joints used as replacements for synovial joints do not contain articular cartilage, and therefore components with different properties towards improving the tribological properties in artificial joints are needed. The present invention provides compositions comprising lubricants that are able to adsorb onto the surfaces (i.e. boundary lubrication) of the artificial orthopaedic implants to manifest their lubricating capabilities in vivo, as well as being capable of having a lubricating effect in an environment that resembles that of synovial fluid.

Thus, an aspect of the present invention relates to an artificial joint implant comprising
a first artificial element 1 and a second artificial element 2 constituting an artificial joint arranged for being implanted in a subject;
a lubricant compartment 3 between said first element and said second element, said lubricant compartment comprising one or more amphiphilic polymer lubricants in a liquid state serving to reduce tribological friction and wear of said implant.

Preferably the amphiphilic polymer is a co-polymer. More preferably the co-polymer is a triblock copolymer such as F127, F108, F68, and P105. From Example 4+FIGS. 3-5 it can be seen that the co-polymers have a much higher COF compared to natural polymers but also compared to homopolymers such as PEG/PEO. The experiments performed in example 4 resembles the state in an implanted joint implant very closely since the control condition is a model synovial fluid and not water as disclosed in the Honda et al. publication.

It is to be understood that the wording “serving to reduce tribological friction and wear of said implant” may also be formulated as “serving to reduce aseptic loosening of the implant after implantation”.

As previously mentioned artificial joint implants may use synovial fluid as a lubricant simply due to the fact that the synovial fluid may enter the lubricant compartment. Thus, in an embodiment said lubricant compartment 3 is arranged for being in fluidic contact with synovial fluid after implantation.

The lubricant compartment may be arranged for receiving the lubricant both before and after implantation. Thus, in an embodiment the artificial joint implant is arranged for refilling of the lubricant compartment with an polymer lubricant after implantation in a subject. Such arrangement may be that the implant has an easy access for a syringe to the lubricant compartment.

It may be an advantage that the lubricant is not coated on the implant but is in a liquid state. Any coating has a limited lifetime, and especially the coatings made of organic layers under tribological stress are very easily rubbed of. On the contrary, liquid-state lubricant can be continuously supplied from external resources as shown in this invention. In addition, since coating-style lubricants should be integrated into implant manufacturing process, only the patients who will receive surgery in the future can potentially benefit, whereas liquid-state lubricants can be applied to any individual with implants. Furthermore, since the liquid-state lubricants are entirely independent from both manufacturing and surgery, any potential improvements in the future can be immediately applicable to any individual with implants. Thus, in an embodiment the polymer lubricant is not covalently linked and/or not coated to a surface of the first artificial element 1 or the second artificial element 2.

In an additional embodiment the artificial joint implant comprises an element or chamber capable of releasing lubricant into the lubricant compartment 3. In yet an embodiment the element or chamber capable of releasing lubricant into the space is positioned in the first artificial element and/or the second artificial element. The release of lubricant may be electronic release or mechanical release. In yet another embodiment said artificial joint implant comprises an opening for positioning/admistering lubricant in the element or chamber capable of releasing lubricant into the lubricant compartment 3. An advantage of such release chamber is that the patient does not require as regularly having lubricant administered to the artificial joint from an ex vivo source.

### Method for Preparing an Artificial Joint Implant

The implants according to the present invention may be prepared in different ways. Thus, an aspect of the present invention relates to a method for preparing an artificial joint implant comprising
providing an artificial joint implant comprising
   a first artificial element 1 and a second artificial element 2 constituting an artificial joint arranged for being implanted in a subject;
   a lubricant compartment 3 between said first element and said second element;
positioning one or more amphiphilic polymer lubricants in a liquid state in the lubricant compartment 3; and
providing an artificial joint implant according to the present invention.

As also mentioned above, preferably the amphiphilic polymer is a co-polymer. More preferably the co-polymer is a triblock copolymer such as F127, F108, F68, and P105. From Example 4+FIGS. 3-5 it can be seen that the co-polymers have a much higher % reduction in COF compared to natural polymers but also compared to homopolymers such as PEG/PEO.

The positioning of the one or more amphiphilic polymer lubricants in a liquid state in the lubricant compartment 3, may take place at different points in time. Thus, in an embodiment the method is performed ex vivo.

In yet another embodiment the one or more amphiphilic polymer lubricants are positioned in the lubricant compartment after an artificial joint implant has been implanted in a subject.

### Medical Use of Lubricant

As described earlier, the pharmaceutical compositions of the present invention comprises one or more lubricants, where the one or more lubricants are capable of improving the tribological properties of artificial orthopaedic post-surgery, preferably by injecting the lubricants into, or to the proximity of an artificial joint in an individual having one or more artificial implants. Thus, in a further aspect the present invention pertains to lubricants according to the present invention for use as a medicament.

In in yet a further aspect, the present invention relates to a lubricant as described herein for use in preventing and/or treating and/or alleviating conditions associated with an orthopaedic implant.

Conditions associated with orthopaedic implants may be any condition that is related to the implantation of the artificial joint, and subsequently functioning of the implant. Since wear of the implant may result in the release of wear particles from the surfaces of the implant to the surrounding environment, examples of such conditions may be infections, inflammatory conditions, tissue damage, internal haemorrhage, osteolysis, acetabular loosening and dislocation, poisoning such as metal poisoning, and any other conditions arising successive to these conditions.

In particular, the present invention relates to a lubricant for use in preventing and/or treating and/or alleviating conditions associated with the functioning of the artificial orthopaedic implant, such as increased friction and wear, i.e. tribological wear of the implant. Hence, the present invention also relates to a lubricant for use in reducing (delaying) tribological wear of said orthopaedic implant. In a further embodiment, said orthopaedic implant is a UHMWPE versus CoCrMo alloy implant, a UHMWPE versus alumina ceramic implant, a CoCrMo alloy versus CoCrMo alloy implant, or a alumina ceramic versus alumina ceramic implant.

Since the lubricant of the invention is suitable for application to artificial joints that are already implanted in an individual, the lubricant is preferably provided in a form suitable for delivery into said individual. Therefore, the present invention relates to a lubricant for use as a medicament or for use in preventing and/or treating and/or alleviating conditions associated with an orthopaedic implant, where said lubricant is provided in the form of a composition such as a pharmaceutical composition as described herein. Additionally, the present invention relates to a lubricant as described herein for use as a medicament. In particular the present invention relates to a lubricant as described herein for use in preventing and/or treating and/or alleviating conditions associated with an orthopaedic implant, where said lubricant is formulated for administration by injection such as for injection into or in the proximity of said orthopaedic implant, for example for injection into the interface of the acetabular cup and femoral head of an implant.

The lubricant may be the same as the one or more lubricants comprised in the pharmaceutical compositions according to the invention, and hence the present invention relates in one embodiment to a lubricant as described herein for use as a medicament or for use in preventing and/or treating and/or alleviating conditions associated with an orthopaedic implant, where said lubricant is a synthetic lubricant or a natural lubricant.

In another embodiment, the present invention relates to a lubricant for use as a medicament or for use in preventing and/or treating and/or alleviating conditions associated with an orthopaedic implant (such as aseptic loosening), where the lubricant is an amphiphillic copolymer.

In yet another embodiment, the present invention relates to a lubricant for use as a medicament or for use in preventing and/or treating and/or alleviating conditions associated with an orthopaedic implant, where the lubricant is selected from one of the groups consisting of poly(ethylene oxide)-based block copolymers, Carbohydrate-based block copolymers, Poly(vinylpyrrolidone)-based block copolymers, and alpha-acid glycoproteins.

Accordingly, the present invention also relates to a lubricant for use as a medicament or for use in preventing and/or treating and/or alleviating conditions associated with an orthopaedic implant, where the lubricant is selected from the group of di-block copolymers consisting of poly(propylene oxide)-b-poly(ethylene oxide), poly(butadiene(1,2 addition))-b-poly(ethylene oxide), poly(butadiene(1,4 addition))-b-poly(ethylene oxide), poly(ethylene oxide)-b-poly(s-caprolactone), poly(ethylene oxide)-b-polylactide), poly(ethylene oxide)-b-poly(methyl acrylate), polyisobutylene-b-poly(ethylene oxide)-b-Poly(Isobutylene), and pPoly(ethylene oxide)-b-poly-Lactide, poly(ethylene oxide)-b-poly(acrylic acid), poly(ethylene oxide)-b-poly(acrylamide), poly(ethylene oxide)-b-poly(methyl acrylate), poly(ethylene oxide)-b-poly(N-isopropylacrylamide), poly(ethylene oxide)-b-poly(dimethylsiloxane), poly(butadiene(1,2 addition)-b-poly(acrylic acid), poly(butadiene(1,4 addition))-b-poly(acrylic acid), poly(Isobutylene)-b-poly(acrylic acid), Poly(vinyl pyrrolidone)-b-Poly(D/L-Lactide), and tri-block (A-B-A) forms of the above mentioned di-block copolymers.

In an embodiment, the present invention relates to a lubricant for use as a medicament or for use in preventing and/or treating and/or alleviating conditions associated with an orthopaedic implant, where the lubricant is selected from the group of di-block copolymers consisting of poly(propylene oxide)-b-poly(ethylene oxide), poly(butadiene(1,2 addition))-b-poly(ethylene oxide), poly(butadiene(1,4 addition))-b-poly(ethylene oxide), and any tri-block copolymeric forms (A-B-A or B-A-B) of the above mentioned di-block copolymers. A diblock copolymer composed of PEO and PPO only, may have similar effect with the confirmed triblock copolyemers in this work.

In another embodiment, the present invention pertains to a lubricant for use as a medicament or for use in preventing and/or treating and/or alleviating conditions associated with an orthopaedic implant, where the lubricant is selected from the group of di-block copolymers consisting of poly(ethylene oxide)-b-poly(s-caprolactone), poly(ethylene oxide)-b-poly-Lactide, poly(ethylene oxide)-b-poly(methyl acrylate), poly(ethylene oxide)-b-poly(Isobutylene), poly(ethylene oxide)-b-polyLactide, poly(ethylene oxide)-b-poly(acrylic acid), poly(ethylene oxide)-b-poly(acrylamide), poly(ethylene oxide)-b-poly(methyl acrylate), poly(ethylene oxide)-b-poly(N-isopropylacrylamide), and any tri-block copolymeric forms (A-B-A or B-A-B) of the above mentioned di-block copolymers.

In yet another embodiment, the present invention relates to a lubricant for use as a medicament or for use in preventing and/or treating and/or alleviating conditions associated with an orthopaedic implant, where the lubricant is selected from the group of di-block copolymers consisting of di-block copolymers consisting of poly(ethylene oxide)-b-poly(dimethylsiloxane), poly(butadiene(1,2 addition))-b-poly(acrylic acid), poly(butadiene(1,4 addition))-b-poly(acrylic acid), poly(Isobutylene)-b-poly(acrylic acid), Poly(vinyl pyrrolidone)-b-Poly(D/L-Lactide), and any tri-block copolymeric forms (A-B-A or B-A-B) of the above mentioned di-block copolymers.

It is within the scope of the present invention that the lubricant described above may be selected independently from any of the different groups presented above. Thus, the lubricant described herein may be applied as a single species of lubricant or in a composition of lubricants such as a composition of two or more lubricants, for example three, four or five independently selected lubricants. The lubricants in a composition may be present in equal amount or the amounts of the individual lubricants in the composition may vary.

### Method of Treatment

In yet a further aspect, the present invention does also relate to a method of preventing and/or treating and/or alleviating a condition associated with an orthopaedic implant, said method comprising a step of administering to a subject in need thereof an therapeutically effective amount of a pharmaceutical composition comprising one or more lubricants according to the present invention.

In one embodiment the method relates to a condition associated with an orthopaedic implant, where said condition is tribological wear of said orthopaedic implant.

In another embodiment the method relates to a pharmaceutical composition comprising one or more lubricants according to the invention, where said composition is administered by injection into said implant or to the proximity of said implant.

In yet another embodiment the method relates to one or more orthopaedic implants, where the orthopaedic implant is selected from the group consisting of a hip prosthesis, a knee prosthesis, a shoulder prosthesis, a foot and ankle prosthesis, a toe prosthesis, an elbow prosthesis, a hand and wrist prosthesis, a finger prosthesis and any possible combination thereof.

In a further embodiment the method relates to one or more orthopaedic implants, where the orthopaedic implant is a UHMWPE versus CoCrMo alloy implant, a UHMWPE versus alumina ceramic implant, a CoCrMo alloy versus CoCrMo alloy implant, or a alumina ceramic versus alumina ceramic implant. In an embodiment the implant comprises UHMWPE and the lubricant is an amphiphillic lubricant.

The artificial orthopaedic implant may be coated with the pharmaceutical compositions and lubricants of the invention prior to surgery, and the compositions and lubricants may be administered immediately after surgery so as to ensure efficient lubricating conditions from the beginning. Alternatively the lubricant is not coated on the implant but is positioned in a fluidic state in the implant as also shown in the figures. Depending on both the physical-chemical nature of the compositions and lubricants, and the type of artificial orthopaedic implant to be lubricated, administration of the pharmaceutical compositions and lubricants of the invention may be performed in various time-intervals. It is within the scope of the invention that the pharmaceutical compositions and lubricants may be administered at any time when it is established, that a better lubricating effect is needed in order to reduce wear of the artificial orthopaedic implant.

However, it is also within the scope of the invention that pharmaceutical compositions and lubricants may be administered on a regular basis, so as to ensure that the artificial orthopaedic implant is sufficiently lubricated at all times. Hence, the pharmaceutical compositions and lubricants according to the invention may be administered on a daily basis or every second day, or on a weekly basis such as 1-3 times every week. In cases of effective lubrication by the pharmaceutical compositions and lubricants of the invention, it may only be necessary to administer the compositions and lubricants every second or third week or every month to ensure sufficient lubrication of the artificial orthopaedic implant. Some of the pharmaceutical compositions and lubricants according to the present invention may have profound lubricating effects, and it may therefore only be necessary to administer these 1-6 times every year or every second year, in order to ensure sufficient lubrication of the implant.

The pharmaceutical compositions and lubricants of the present invention will have lubricating effects to various degrees. Therefore, the amount of composition and lubricant to be administered each time will depend on the physical-chemical nature of the compositions and lubricants, as well as on the type of artificial orthopaedic implant to be lubricated. Therefore, when the pharmaceutical composition and lubricant according to the invention have very effective lubricating properties, and/or the size or type of the implant requires only a relative small amount of lubricant, the pharmaceutical compositions and lubricants according to the invention may be administered in relatively small aliquots every time of administration, such as 1-10 μl, such as 1-5 μl, such as 1-4 μl, or such as 1-3 μl; for example approximately 2 μl, or approximately 5 μl or approximately 8

Some pharmaceutical compositions and lubricants may be administered in larger amounts, such as in aliquots of 10-100 μl per administration, for example 25 μl, such as 50 μl or for example 75 μl per administration, depending on the size and/or type of artificial orthopaedic implant to lubricate. Other compositions and lubricants may be administered in even larger amounts per administration, e.g. in aliquots of 100-1000 μl, such as 250 μl, for example 500 μl or 750 μl, again depending on the size and/or type of implant to lubricate.

Some orthopaedic implants, e.g. a knee or a hip implant, may require even larger amounts of the pharmaceutical compositions and lubricants of the present invention per administration, such 1-10 ml, for example 2.5 ml, or 5 ml or 7.5 ml per administration, depending on the nature of the composition and lubricant to be administered. Preferably an amount in the range 0.5-3 ml such as 1-2 ml is administered.

It is within the scope of the present invention that the pharmaceutical compositions, lubricants, methods and uses are suitable for any animal having one or more artificial implants, in particular any mammal and most particular a human being.

It should be noted that embodiments and features described in the context of one of the aspects of the present invention also apply to the other aspects of the invention.

### EXAMPLES

### Example 1

### Pin-On-Disk Tribometry method

The conditions of the experiments were as follows: (a) Instrument: Pin-on-disk tribometry, pure sliding contacts (b) Tribopair: CoCrMo pin (flat-ended with the diameter of 7.1 mm) and high density polyethylene (HDPE) disk (30 mm in diameter, 5 mm in thickness) (c) the load=10 N (d) sliding speed=1 mm/s (f) temperature=ca. 25° C. (room temperature) (g) model synovial fluid=Commercial calf serum (Hyclone SH30212.03 Alpha Calf fraction)

The lubricant of the present invention: the lubricant solution was prepared by dissolving Pluronic F105 (PEO-PPO-PEO, tri-block copolymer) at high concentration (20% or 200 mg/ml) in HEPES buffer solution (pH 7, aqueous buffer). 1 ml aliquot of the lubricant of the present invention was injected using needle/syringe into the tribocup, where the sliding contact between the CoCrMo pin against HDPE is already taking place in fluids (19 ml). The final concentration of the lubricant of the present invention in the tribocup was about 1% or 10 mg/ml.

The lubricant was P105 with the final concentration 1%—due to much larger volume of the tribocup in this example (15 ml) than the others (3 ml), and due the design, it is speculated that the lubricants, P105, have not effectively reached on the UHMWPE surface. Optimized experiments are e.g. provided in example 4.

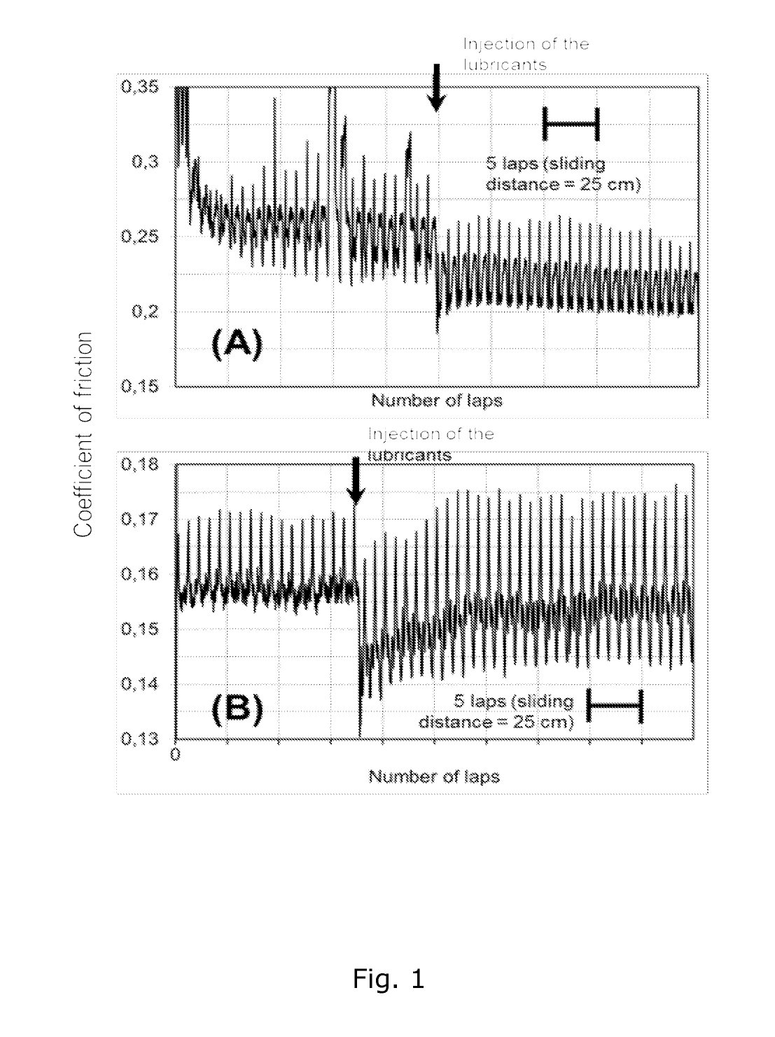

In FIG. 1 (A), the fluid is HEPES buffer solution. Immediately after the injection of the lubricant of the present invention, the reduction in the coefficient of friction occurs by ca. 0.05 (from approximately 0.27 to 0.22), and this lower value were maintained until the end of measurements. This experiment shows that the aqueous solution containing the lubricant of the present invention at this concentration lowers the coefficient of friction in neutral aqueous environment.

In FIG. 1 (B), the fluid is calf serum, serving as model synovial fluid. Since the fluid is different, the coefficient of friction before injecting the lubricant of the present invention is already different, and the coefficient of friction is slightly lower than 0.16. Upon injection of the lubricant of the invention, the coefficient of friction dropped immediately by approximately 0.015. Furthermore, lower frictional properties persisted for at least 10 laps.

The results are show that despite the presence of many different biomolecules in calf serum which is a model for synovial fluid, the lubricant of the present invention still showed lubricating effect.

Testing of the various lubricants of the present invention at CoCrMo alloy-vs.-UHMWPE pair, alumina ceramic-vs.-UHMWPE pair, CoCrMo alloy-vs.-CoCrMo alloy pair, and alumina ceramic-vs.-alumina ceramic were also carried out. The focus was firstly placed on characterizing the magnitude of reduction in friction forces upon injection of the lubricants into the liquid cell where the sliding contacts between the tribopairs are in progress. Three types of model synovial fluids will be used for screening tests;

Model synovial fluid (1): serum solution

Model synovial fluid (2): serum solution+hyaluronic acids

Model synovial fluid (3): synovial fluid

The serum and synovial fluid are obtained from animal and/or commercial resources.

The testing parameters for the tribometer, including load, sliding speed, number (distance) of laps, and the contacting geometry, were optimized during the experimental phase. Likewise, the concentrations of the lubricants of the present invention were optimized during the experimental phase.

Additionally, wear tests were performed to characterize the magnitude of reduction in wear properties upon injection of the lubricants of the present invention into the liquid cell where the sliding contacts between the tribopairs are in progress.

Quantitative characterization of wear properties were also conducted by gravimetric methods (i.e. by weighing the tribopairs, before and after the tribostress, and with or without the lubricants of the present invention). In parallel, qualitative characterization of wear properties were conducted by optical microscopy (particle shape, size, and distribution).

### Example 2

### Joint Simulator Method

### Hypothetical Example

The tests of friction and wear properties of the lubricants of the present invention are carried out according to the Joint Simulator method using a Hip and Knee Joint simulator.

The Joint simulator method are similar to the Pin-On-Disk Tribometry method, but is more realistic since real hip/knee joint implants are employed as the articulating surfaces (for all pairs). The lubricant molecules of the present invention revealing high potential screened from Pin-On-Disk Tribometry experiments are tested. Animal serum solutions and synovial fluids are employed as model synovial fluid. The efficacy of the lubricant molecules to reduce the friction and wear by the addition of the lubricants of the present invention will be tested over at least one million articulations. Standard strain gauge will provide the change of coefficient of friction (COF), and volumetric/gravimetric approaches will provide the change of wear properties.

### Example 3

### Materials

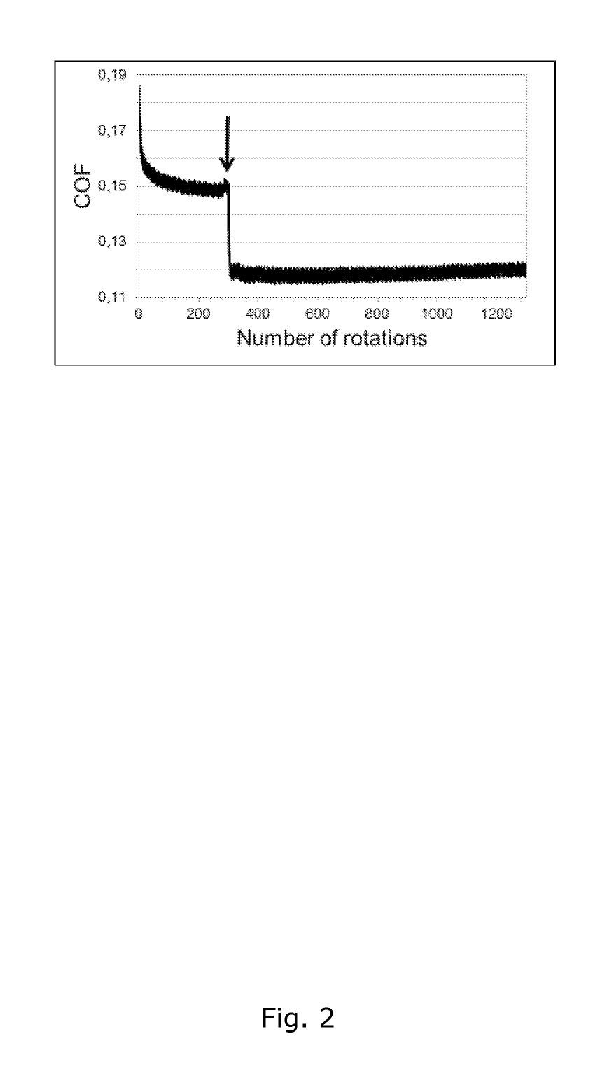

F127 was dissolved in HEPES buffers solution (pH 7, 1 mM HEPES, no extra salts) at the concentration of 20% (=200 mg/ml), and the lubricating effects at the sliding contacts between CoCrMo/UHMWPE in calf serum were assessed adding F127 into serum by means of Pin-on-Disk Tribometry. The results are shown in the FIG. 2.

### Method

In this experiment, first, the sliding contact between CoCrMo/UHMWPE started in calf serum (2 ml) under 10N and at the speed of 10 mm/s. The COF decreased rapidly in the beginning ca. 100 rotations (=“running-in” process), but became stabilized at ca. 0.15 thereafter. At the 300^{th} rotation, 1 ml of the F127 solution was injected into the tribocup where the sliding contacts between CoCrMo/UHMWPE is taking place. Thus, the final concentration of F127 in calf serum is 6.7% w/v (67 mg/ml). The COF dropped immediately to ca. 0.12 and this value maintained until the end of the measurements. The total number of rotations after the injection of the F127 20% was 1,000, and this corresponds to 49 m in total length (the F127-lubricated contact length is ca. 38 m).

### Results

Repeated experiments (6 times) under the same condition have shown that a reduction of COF always occurred under the same condition. However, absolute COF values before injection, as well as those after injection, are not identical for each measurement, the evaluation of lubricating effect by the lubricant was carried out by calculating the % Reduction of COF upon injection of the lubricant.

% Reduction in COF=((COF(before injection)−COF(after injection)/(COF (before injection))×100%

For example, if the COF changes from 0.2 to 0.1 upon injection of a lubricant, % Reduction in COF=((0.2−0.1)/0.2)×100%=50%, and it means that the lubricant has a capacity to reuce the friction coefficient to its a half of the unlubriated contact.

### Conclusion

The result shown in FIG. 2 indicates that the injection of 1 ml of 20% F127 in HEPES into calf serum immediately reduces the COF of CoCroMo/UHMWPE by 20%, and this reduced COF persisted until the end of the test (1000 rotations or 38 m).

### Example 4

### Methods

The same tests as described under example 3 were extended to a number of other lubricants, including (a) amphiphilic triblock copolyemrs, F127, F108, P105, and F68, (b) homopolymers, PEO 5k, PEO 600k, and PAA 5.1 k, (c) biological polymers, Hyaluronic Acid (HA), bovine submaxillary mucin (BSM), bovine serum albumin (BSA), and Alginic Acids (AA). The concentration of the lubricants was also varied to 40%, 20%, 10%, 5%, 3%, 1%, and 0.1%. The range of concentration was primarily determined by the solubility of each lubricant in HEPES buffer solution; some lubricants, e.g. PEO 5k, showed extremely high solubility (up to 40% in this tests, but could be higher), whereas some other lubricant, PEO 600k, showed very low solubility (maximum 5% in this test). The tribological conditions, load (10 N), speed (10 mm/s), the number of rotations (300 rotation before the injection and 1,000 rotations after the injection), the total length of tribological stress (49 m), and finally the method of evaluation remained the same with the case of F127 20%. It is noted again that the final concentration of the lubricants in calf serum after injection is ⅓ of the original composition of the lubricant.

Lower concentrations of the biological polymers were because of poor solutbility. HA and AA were too viscous and gel-like to dissolve further. BSM and HA are better dissolved, and can be compared to the copolymers with the corresponding concentrations.

### Results and Conclusions

### 1. Copolymers (Pluronics)

The test results of the copolymers are shown in FIGS. 3 and 4.

All four copolymers, F127, F108, P105, and F68, showed significant reduction in COF upon injection of 1 ml of the lubricant into 2 ml of calf serum. The magnitude of reduction in COF, as expressed by % reduction in COF, is generally proportional to the concentration, and the maximum % reduction in COF lies between 20-25%. The results are shown in FIG. 3.

### 2. Homopolymers

All three homopolymers, PEO 5k, PEO 600k, and PAA 5.1 k, also showed reduction in COF upon injection of 1 ml of the lubricant into 2 ml of calf serum. Due to high solubility of PEO 5k in HEPES buffer, the concentration of PEO 5k was tested up to 40%. On the other hand, due to poor solubility of PEO 600k in HEPES buffer, the concentration of PEO 600k was tested up to 5% only. The results are shown in FIG. 4.

For the same concentration, the % reduction in COF was clearly smaller than those of the copolymers, since the maximum % reduction in COF is not higher than 10%.

### 3. Biological Polymers (Natural Polymers)

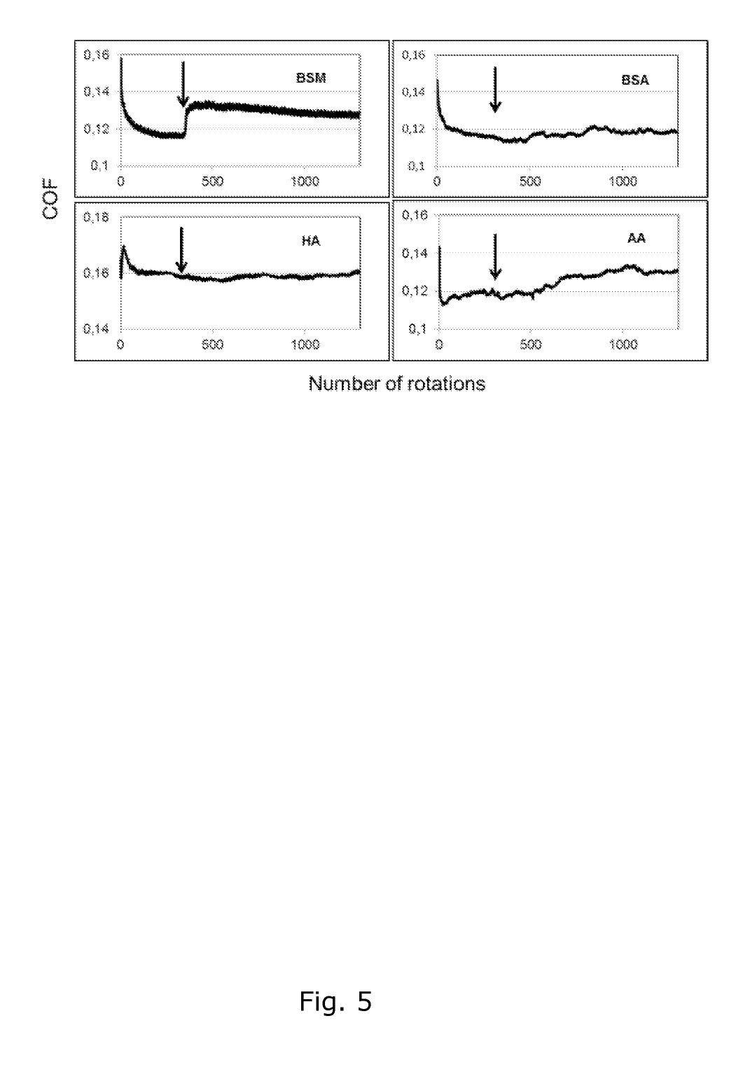

Four biological polymers were tested, Bovine Submaxillary Mucin (BSM), Hayluronic Acid (HA), Bovine Serum Albumin (BSA), and Alginic Acid (AA). They did not show any noticeable reduction in COF upon injection. The results are shown in FIG. 5.

No biological polymers employed in this test showed noticeable reduction in COF upon injection. BSM showed rather an increase of COF upon injection. It is of particular interest to note that HA does not show any reduction in COF since it is one of the major components of natural synovial fluid and the major composition of viscosupplementation, the medicament to be injected towards defected natural articular joints, such as osteoarthritits. This observation confirms again that the nautral component of synovial fluid is not sufficient to further lubricate the artificial joints and synthetic lubricants that are ideally suited for artificial implants are necessary for more effective lubrication.

### Example 5

### Influence of Temperature and Hyaluronic Acids Model Synovial Fluid (=Physiologically More Relevant Condition)

### Methods

In order to test the lubricating effect by the lubricants in physiologically more relevant environment, two parameters, temperature and the presence of hyaluronic acid in calf serum, were adjusted.

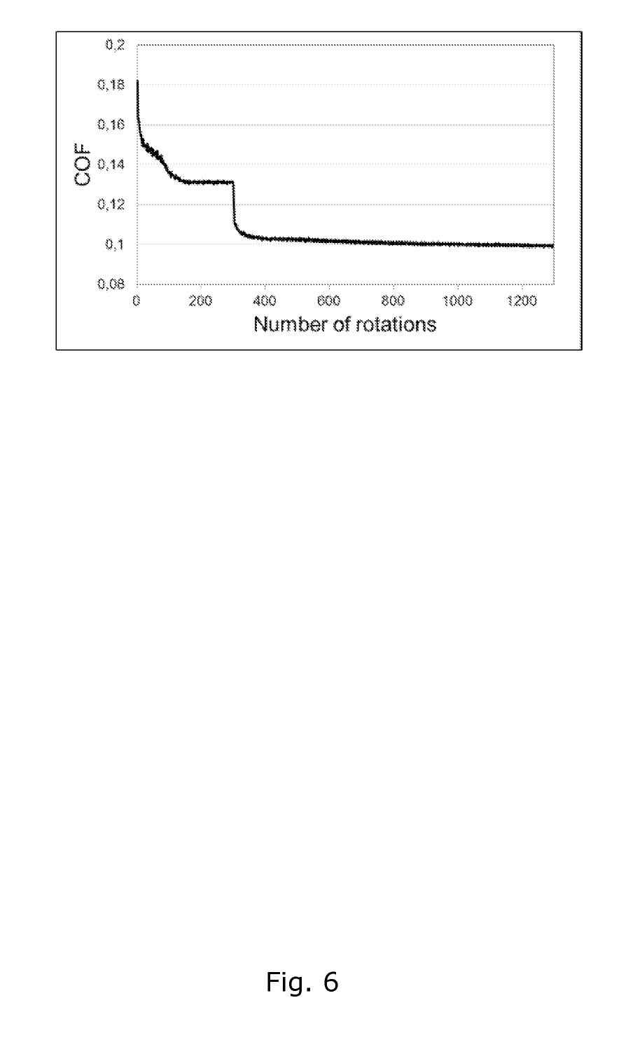

Firstly, the lubricating effect of F127 20% at body temperature (37° C.) was assessed by means of Pin-on-Disk tribometry. Except for the temperature, the experimental conditions are identical with those with FIG. 2. The results are shown in FIG. 6.

The % reductions in COF of F127 20% obtained under 22° C. (19.7%) and 37° C. (23.1%) are nearly the same. This result validates that the observations made at 22° C. are also valid at physiological temperature.

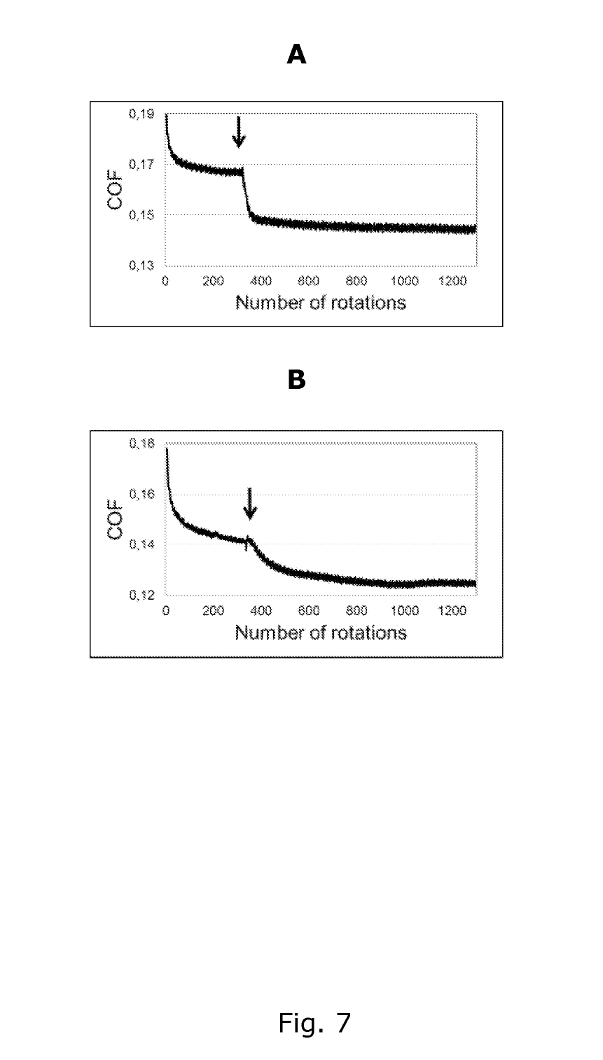

Secondly, HA was added to calf serum to further emulate synovial fluid. Apart from the concentration of proteins, the presence/absence of HA is the major difference between synovial fluid (HA present) and serum (HA absent). HA was dissolved in calf serum at the concentration of 3.5 mg/ml, which falls in the range of HA concentration of healthy synovial fluid. This calf serum is called “HA-serum” in the following. F127 20% and F127 10% were tested with HA-serum, and the results are shown in FIG. 7.

For both F127 20% and 10%, the % reduction in COF were slightly smaller for the case of HA-serum (13.4%) compared to serum alone (18.5% on average) for F127 20%, and HA-serum (11.9%) compared to serum (15.7% on average) for F127 10%. For F127 10%, it takes somewhat longer for the COF to re-established at a lowered value. This is probably due to that heavy HA is blocking the adsorption of the lubricant molecules to a certain extent. Howvever, the duration of establishing time is only 5-10 minutes, and for the long-run, the influence of HA appears to be negligible.

### Example 6

### Long-Term Tests

### Methods

Long-term tests were carried out at two different speeds, 10 mm/s and 100 mm/s. The load was increased to 20 N. At 10 mm/s, the test was extended to 10,000 rotations, which corresponds to ca. 300 m in total sliding distance. At 100 m/s, slower speed covered 100,000 rotations, which corresponds to ca. 3,000 m in total sliding distance. Both measurements take roughly 8 and ½ hours to complete.

### Results

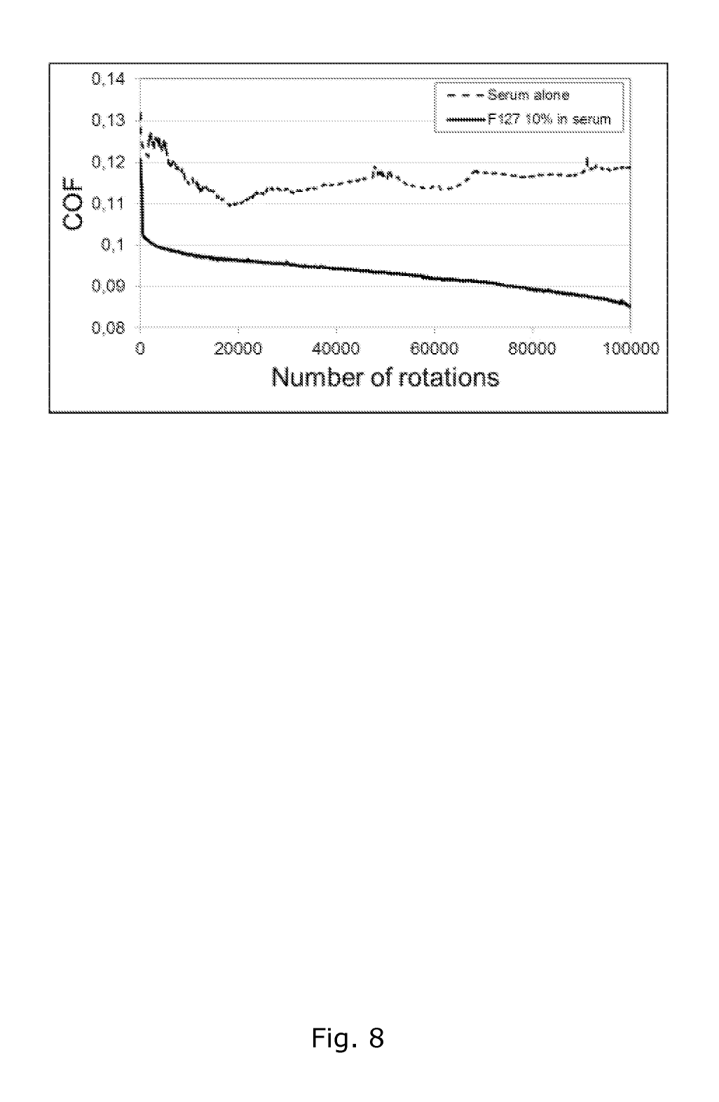

A representative example of F127 10% and serum alone under 100 m/s is shown in FIG. 8. After injection of the F127 10%, lowered COF continued until the end of the measurements. This long-term lubricating effect was tested with F108 10%, F68%, and P105 10%, and all of them showed similar, long-term lubricating effect.

### Conclusion

The results in FIG. 8 suggest that the lubricating effect by the 4 copolymers is likely to be happening much longer periods than shown in this test.

### Example 7

### Aged or Damaged Tribopairs

All the tribopairs employed for the previous tests were freshly prepared; after turning of UHMWPE disks, they were rinsed with distilled water, ethanol, followed by nitrogen blow dry. An advantage of the current invention is the prevention/reduction of the wear of implant materials post-surgically, e.g. by injection. To confirm this, standard tests were carried out on model implant materials that were treated differently to represent “aged” or “damaged” implants.

### Methods

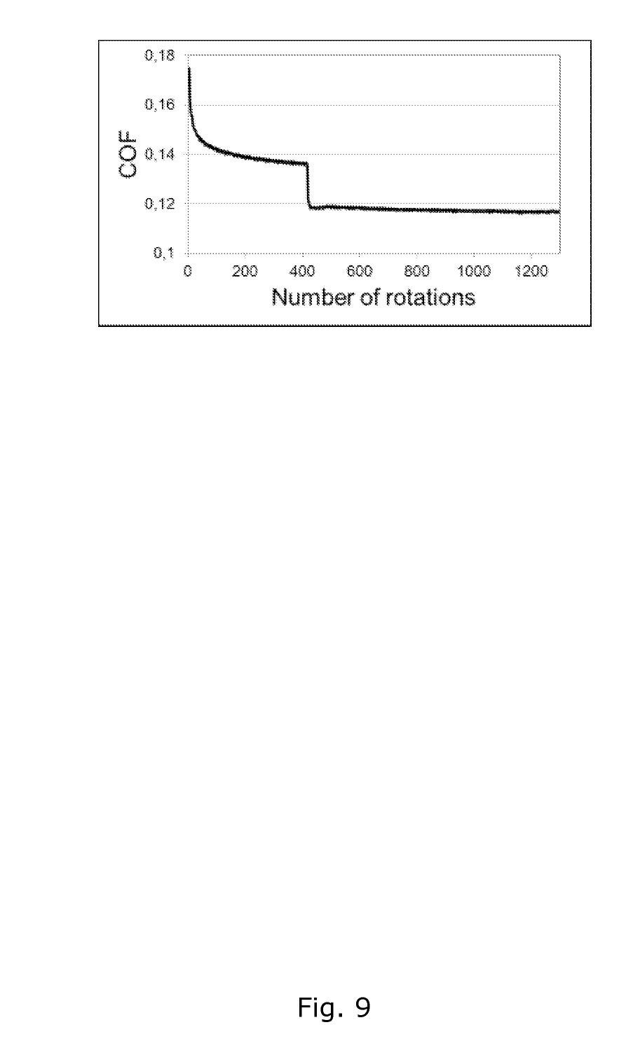

“Aged” or “damaged” implants were made in 3 different ways (1) surface roughening (2) exposure to UV/03 (3) soaking in calf serum for 4 weeks; (1) Surface roughness was made either polishing with sandpaper (P80=average particle diameter of 201 μm) or randomly grinding with a saw on UHMWPE surfaces. (2) Exposure to UV/03 is expected to oxidize the surface of UHMWPE surfaces and the exposure time was 30 minutes (3) Soaking of both UHMWPE and CoCrMo in calf serum (4 weeks) represent the already implanted joints, but no mechanical stress was given. A representative example of “aged tribopair for 4 weeks” is shown in FIG. 9.

### Results

The % reduction in COF was measured to be 14%, and this was nearly the same with the % reduction in COF obtained from freshly prepared tribopairs. For all the other cases, the lubricating effect was observed to be similar with the freshly prepared UHMWPE samples.

### Conclusion

These tests confirm that the lubricating effect by the lubricant is applicable not only to freshly implanted surfaces, but also aged and/or damaged implanted surfaces.

### Example 8

### Cell Viability Tests

### Methods

Cell viability towards the external lubricants were tested by carrying out standard MTT tests on four copolymer lubricants, F127, F108, P105, and F68.

The lubricants were provided at a concentration of 50 mg/ml (5%), and this corresponds to the concentration of 15% prior to injection.

### Results

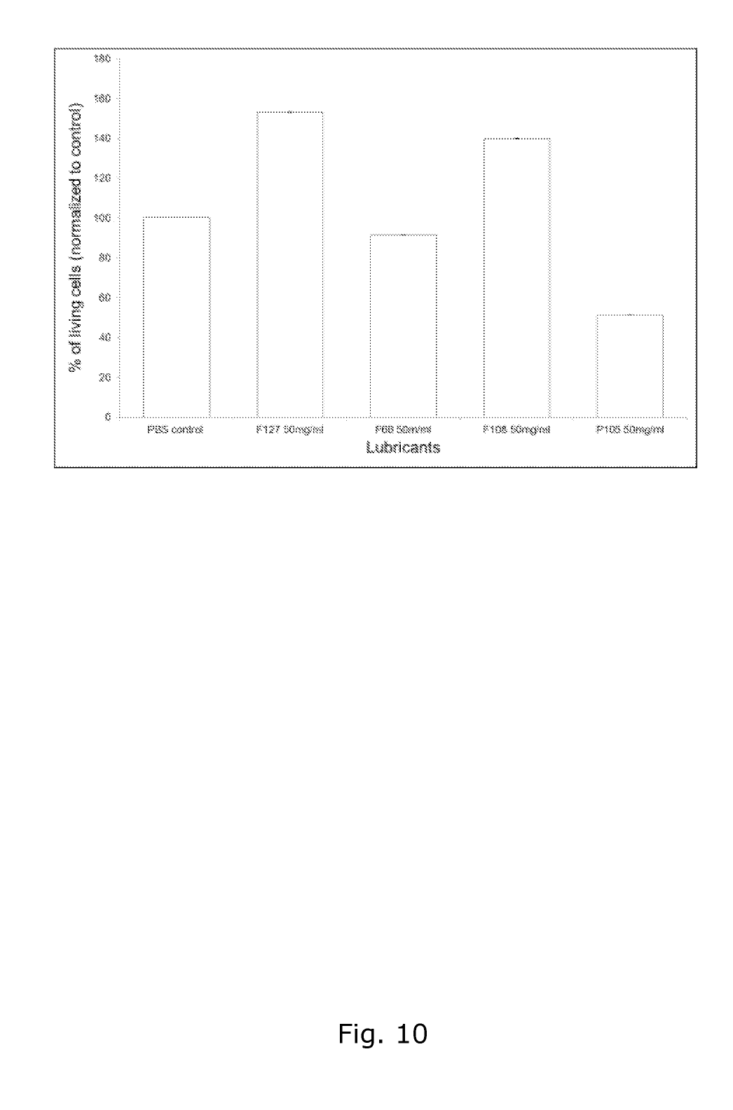

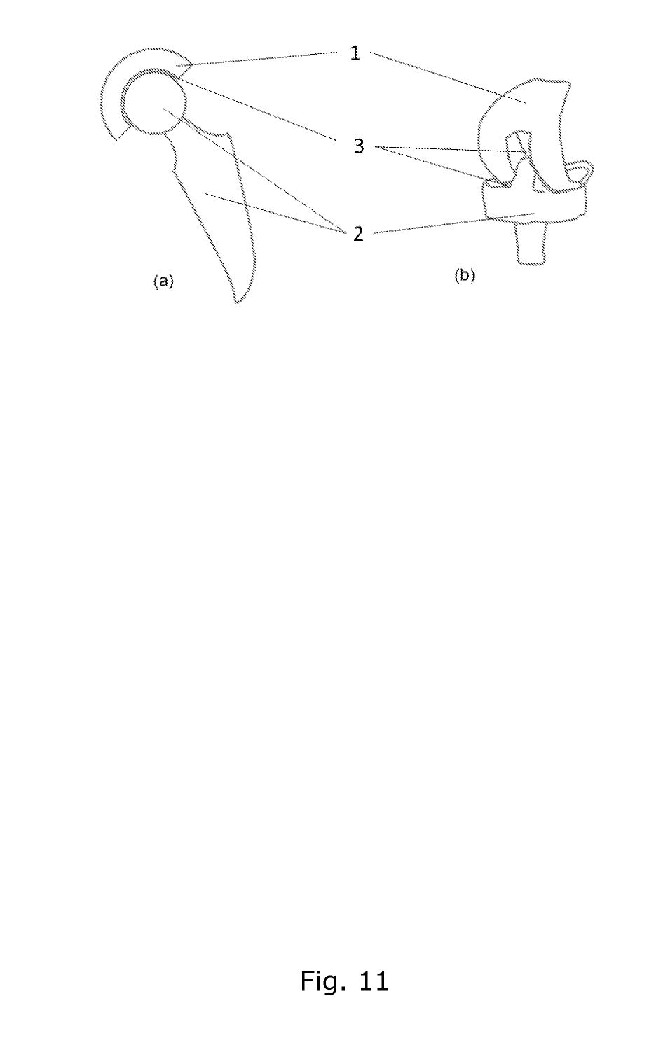

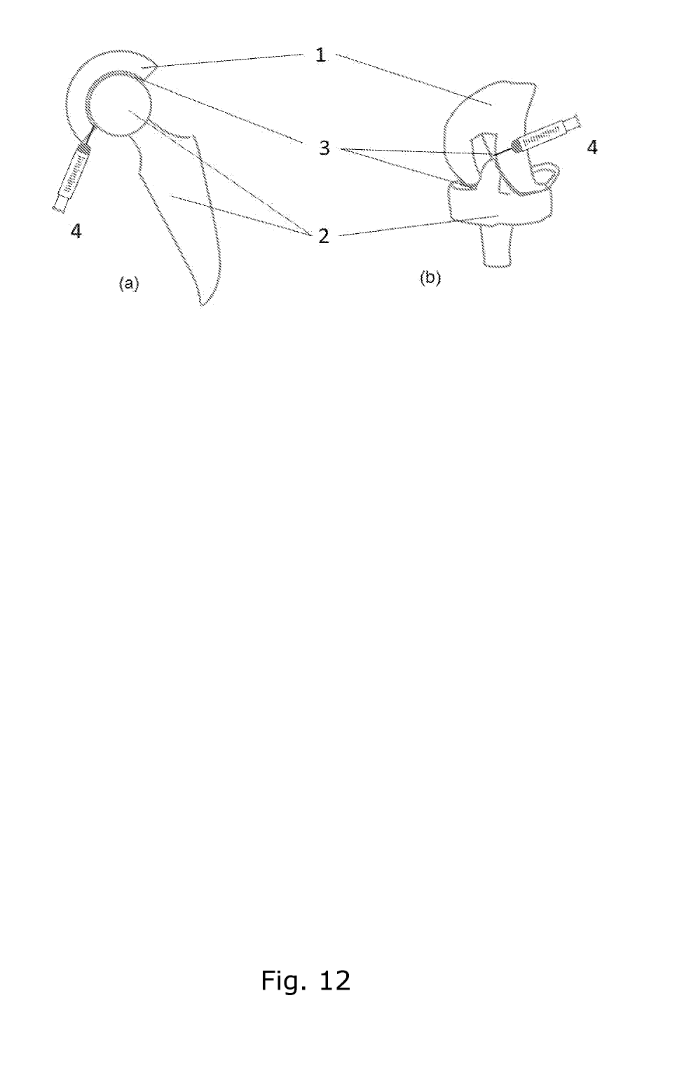

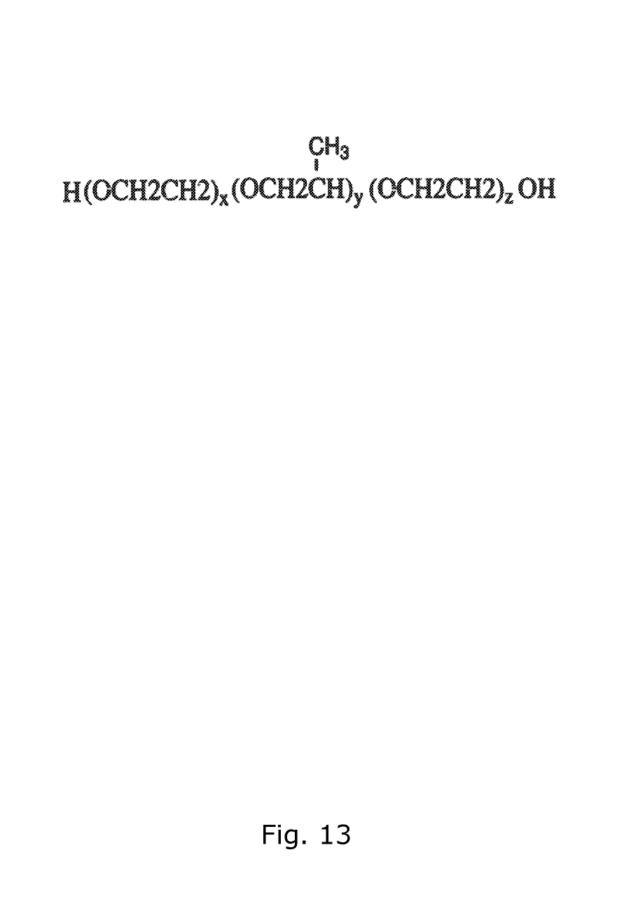

Results are shown in FIG. 10. The first column is for PBS control (=no external lubricants), and the data for the external lubricant are normalized to the control.

### Conclusion

According to this test, F127 and F108 are superior to the others in terms of cell viability (or proliferation), whereas P105 is least in its safety towards fibroblast cells.

1-24. (canceled)

25. A pharmaceutical composition comprising one or more amphiphilic polymer lubricants and at least one pharmaceutically acceptable carrier, for the prevention or reduction of aseptic loosening in a subject comprising an artificial joint implant,
wherein the one or more amphiphillic lubricants are
poly(ethylene oxide)-block-poly(propylene oxide)-block-poly(ethylene oxide) (PEO-PPO-PEO) triblock copolymer.

26. The pharmaceutical composition according to claim 25, wherein the amphiphillic lubricants are selected from the group consisting of F127 (poly(ethylene oxide)-block-poly(propylene oxide)-block-poly(ethylene oxide), average weight 12600 Da), F108 (Poly(ethylene glycol)-block-poly(propylene glycol)-block-poly(ethylene glycol), average weight 11680 Da), F68 (Poly(ethylene glycol)-block-poly(propylene glycol)-block-poly(ethylene glycol), Average molecular weight: 8400 Da), and P105 (Poly(ethylene glycol)-block-poly(propylene glycol)-block-poly(ethylene glycol), Average molecular weight: 6500 Da).

27. The pharmaceutical composition according to claim 25, wherein the pharmaceutical composition is administered to a lubricant compartment between a first artificial element and a second artificial element of an artificial joint implant after the joint implant is positioned in the subject.

28. The pharmaceutical composition according to claim 27, wherein the lubricant compartment is in fluidic contact with synovial fluid.

29. The pharmaceutical composition according to claim 25, formulated for administration by injection.

30. The pharmaceutical composition according to claim 25, wherein the amphiphillic lubricants are present in a total concentration 1-30% (weight/volume), such as 1-20%, such as 1-15%, such as 1-10%, such as 1-5%, such as 5-20%, such as 10-20%, or such as 20-30%.

31. The pharmaceutical composition according to claim 25, where said composition is able to reduce the coefficient of friction according to the Pin-On-Disk Tribometry method by at least 1%, such as at least 5% such as at least 10%, such as at least 15%, such as at least 20%, such as at least 25%, or such as at least 30%.

32. The pharmaceutical composition according to claim 25, wherein the artificial joint implant comprises materials selected from the group consisting of UHMWPE (Ultra-high Molecular Weight Polyethylene), CoCrMo alloy, alumina ceramic and alumina alloy, Zirconia-Toughened Alumina (ZTA) and combinations thereof.

33. The pharmaceutical composition according to claim 25, wherein the artificial joint implant is selected from the group consisting of a hip prosthesis, a knee prosthesis, a shoulder prosthesis, a foot and ankle prosthesis, a toe prosthesis, an elbow prosthesis, a hand and wrist prosthesis, and a finger prosthesis.

34. The pharmaceutical composition according to claim 25, wherein the artificial joint implant is a total joint replacement implant.

35. An artificial joint implant comprising
a first artificial element and a second artificial element constituting an artificial joint arranged for being implanted in a subject;
a lubricant compartment between said first element and said second element, said lubricant compartment comprising one or more amphiphilic co-polymer lubricants in a liquid state serving to reduce tribological friction and wear of said implant;
wherein the one or more amphiphillic copolymer lubricants are poly(ethylene oxide) (PEO)-based block copolymers;
wherein the co-polymer lubricant is a tri-block copolymer,
and
wherein said lubricant compartment is arranged for being in fluidic contact with synovial fluid after implantation.

36. The artificial joint implant according to claim 35, being arranged for refilling of the lubricant compartment with an polymer lubricant after implantation in a subject.

37. The artificial joint implant according to claim 35, wherein the co-polymer lubricant is not covalently linked to a surface of the first artificial element or the second artificial element.

38. The artificial joint implant according to claim 35, wherein the tri-block copolymer is selected from the group consisting of F127 (poly(ethylene oxide)-block-poly(propylene oxide)-block-poly(ethylene oxide), average weight 12600 Da), F108 (Poly(ethylene glycol)-block-poly(propylene glycol)-block-poly(ethylene glycol), average weight 11680 Da), F68 (Poly(ethylene glycol)-block-poly(propylene glycol)-block-poly(ethylene glycol), Average molecular weight: 8400 Da), and P105 (Poly(ethylene glycol)-block-poly(propylene glycol)-block-poly(ethylene glycol), Average molecular weight: 6500 Da).

39. The artificial joint implant according to claim 35, wherein the joint implant comprises UHMWPE.

40. A method for preparing an artificial joint implant according to claim 35 comprising
providing an artificial joint implant comprising
a first artificial element and a second artificial element constituting an artificial joint arranged for being implanted in a subject;
a lubricant compartment between said first element and said second element;
positioning one or more amphiphilic co-polymer lubricants in a liquid state in the lubricant compartment; and
providing an artificial joint implant according to claim 35;
wherein the one or more amphiphillic copolymer lubricants are poly(ethylene oxide) (PEO)-based block copolymers; and wherein the co-polymer lubricant is a tri-block copolymer.

41. The method according to claim 40, wherein the method is performed ex vivo.

42. The method according to claim 40, wherein the one or more amphiphilic co-polymer lubricants are positioned in the lubricant compartment before or after an artificial joint implant has been implanted in a subject.
